(19) United States
(12) Patent Application Publication
(10) Pub. No.: US 2008/0269729 A1
(43) Pub. Date: Oct. 30, 2008

(54) UNINTERRUPTED POWER SUPPLY, ESPECIALLY FOR A REFRACTIVE LASER

(75) Inventor: Juergen Ledermann, Jena (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: CARL ZEISS MEDITEC AG, Jena (DE)

(21) Appl. No.: 12/100,070

(22) Filed: Apr. 9, 2008

(30) Foreign Application Priority Data
Apr. 26, 2007 (DE) 102007020564.5

(51) Int. Cl. A61N 5/067; H02J 1/10; H01S 3/00; G01R 31/36

(52) U.S. Cl. 606/5; 307/19; 372/38.04; 702/63; 702/81; 606/10

(57) ABSTRACT

The invention concerns an uninterruptible power supply that is used in particular with a refractive laser. A rechargeable battery is provided, whereby prior to beginning the treatment the capacity of the battery is tested to assure that it is sufficient to safely complete the treatment in the event the voltage supply is interrupted.

### RELATED APPLICATION

The current application claims the benefit of priority to German Patent Application No. 10 2007 020 564.5 filed on Apr. 26, 2007. Said application is incorporated by reference herein.

### FIELD OF THE INVENTION

The invention concerns an uninterruptible power supply that is used in particular with a refractive laser.

### BACKGROUND OF THE INVENTION

The cornea is treated with a refractive laser with the objective of removing, or at least reducing, existing vision problems by altering the optical properties of the cornea. Principally, two procedures have established themselves.

On one hand, the “LASIK” (laser in situ Keratomileusis) procedure, where material is removed from the cornea by means of ablation from its surface that has been exposed by an incision is well known. Generally, excimer lasers are used for this.

A more modern procedure is intrastromal ablation, where preferably cuts are placed within the cornea, using a femtosecond laser, which restrict a preset limit that subsequently can be removed through an opening incision. The areas of the incision are calculated in such a way that the optical properties of the cornea after the removal of the excised volume result in the best possible vision.

It is also known to use a femtosecond laser to make the incision in the cornea needed for the subsequent LASIK by excimer laser.

All these procedures are executed in series, that is to say that the work on the corneal material is realized through small, greatly limited laser spots and these spots are made, one after the other, across the area to be treated.

It is easy to understand that significant problems must be expected if the treatment of the identified area cannot be completed, for example, if by some mistake the laser's power supply is interrupted, because the optically effective area is then left in an undefined condition, for example part of the area has already been treated while others areas are still in their original condition.

In order to address this problem, it was recommended in German patent reference DE 19 727 573 C1 to conduct the procedure following a special pattern in order to guarantee at any point in time that the entire area to be treated would be in approximately the same state of treatment. This allows for an improvement of the vision even if the treatment has ended prematurely.

In general, uninterruptible power supplies using a rechargeable battery are known (e.g., from DE 102 44 608 A1). These are used mostly to safeguard PC servers and large-scale computer systems against voltage fluctuations or total utility failure. When the utility failure occurs, they transmit information to the connected PC server in order to initiate a controlled shutdown of the server's operating system, because the functioning of most operating systems can be permanently damaged by an uncontrolled shutdown; especially data could be contaminated or destroyed through such an incident.

From German patent reference DE 43 33 520 A1 it is known how to determine the load current and, based on that, the inside impedance of the battery during discharging and how to compare it to a preset value in order to obtain information about the condition of the battery. However, it does not disclose a link between this test result to a minimum required capacity determined by the user.

### SUMMARY OF THE INVENTION

In contrast to the method based upon the state of existing technology of avoiding undefined treatment results because of the termination of treatment due to problems with the laser's power supply, the present invention proposes an uninterruptible power supply using a rechargeable battery, whereby the capacity of the battery is tested prior to treatment to assure that it is sufficient to safely complete the procedure in case the power supply is interrupted.

More specifically, the invention concerns an uninterruptible power supply that is used in particular with a refractive laser. A rechargeable battery is provided, whereby prior to beginning the treatment, the capacity of the battery is tested to assure that it is sufficient to safely complete the treatment in the event the voltage supply is interrupted.

According to the invention, it is further proposed to check the capacity of the battery of the uninterruptible power supply prior to beginning with the procedure and to compare it to the total capacity required to complete the entire treatment. In one embodiment, the treatment cannot be started if this test reveals that the capacity of the battery is not sufficient to guarantee the completion of the procedure. In one embodiment, this flawed condition of the battery is signaled to the operator optically or acoustically or in another suitable manner.

Additional advantageous embodiments of the invention are explained by the patent claims.

### BRIEF DESCRIPTION OF THE DRAWINGS

FIG. 1 shows a schematic of one embodiment of a refractive laser system of the present invention.

### DETAILED DESCRIPTION

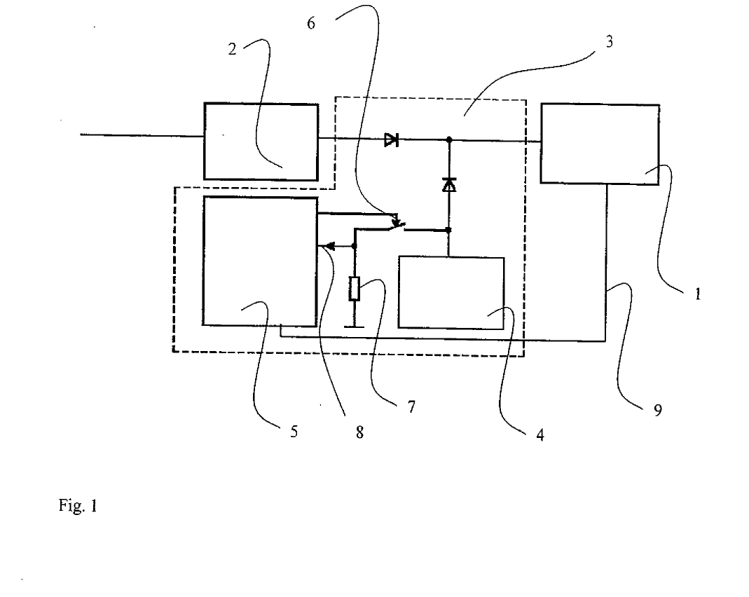

FIG. 1 shows a schematic of a refractive laser system 1, which receives its energy from a power supply module 2. An uninterruptible power supply 3 is placed between the laser system 1 and the power supply 2, which contains a battery 4 and a control module 5.

The control module 5 is able to short-circuit the battery 4 via a load 7 and determine the actual capacity of the battery 4 via a test socket 8. The result of this test is converted into a signal, which is transmitted via a control connection 9 to the refractive laser system 1.

The refractive laser system 1 compares this information about the battery capacity to the requirement for the completion of the entire treatment and permits the initiation of the procedure only if there is sufficient battery capacity.

If this is not sufficient, the procedure does not start and the operator is informed about this in a suitable manner.

This testing of the battery capacity can be conducted either cyclically or just once prior to the beginning of the procedure.

If it is supposed to be done cyclically, the control module will include a pulse generator (not shown here) that defines the cycles.

When determining the required capacity for completing the procedure, the refractive laser system 1 can also consider the existing actual treatment plan. For example, if the planned treatment would take 2 minutes and the average required output of the laser system would be 900 W, then the battery must have a minimum capacity of 0.625 Ah at a battery voltage of 48 V prior to beginning the treatment. If this capacity is not reached, treatment cannot be started.

It will be apparent to one skilled in the art that the design specification of the battery should not be limited to this minimum requirement and an appropriate reserve must be provided, especially to cover peak demands, e.g. when starting up motors. In this case, a battery with a nominal capacity of 10 or 15 Ah would be selected. The battery would be checked prior to starting the treatment to confirm that the minimum capacity (again, with a certain reserve) is still available. Determining the actual capacity by measuring under load is preferable to the conventional testing of the battery voltage, because it could happen that the voltage collapses under load, in spite of the measured nominal voltage in no-load condition, and bridging a utility failure or a defective power supply module would then be impossible and cause the treatment to be unsuccessful.

The invention is not limited to the embodiments described above; instead of being used with the refractive laser system, it could also be used with other loads designed to perform a procedure that must not be interrupted.

1. An uninterruptible power supply for a load with a rechargeable battery, whereby a device is provided to test the battery capacity, characterized by the fact that a device is provided that compares the capacity to the requirement of the load and transmits a signal to the load, which represents the result of this comparison.

2. The uninterruptible power supply according to claim 1, wherein a pulse generator is provided for testing the battery capacity and this device is designed to compare the capacity to the requirement of the load.

3. A refractive laser system for the treatment of a cornea, which incorporates an uninterruptible power supply according to claim 1.

4. A procedure for operating an uninterruptible power supply, comprising:
testing a capacity of a battery;
comparing the tested capacity of the battery to the requirement of the load; and
transmitting the result of the comparison to the load.

5. The procedure for operating an uninterruptible power supply according to claim 4, wherein the testing of the capacity of the battery and the comparison to the requirement of the load occur cyclically.

6. The procedure for operating an uninterruptible power supply according to claim 4, wherein prior to starting a device that is not to be interrupted, the testing of the battery capacity and the comparison to the requirement of the load are carried out.

7. Application of the procedure according to claim 4 with a refractive laser system.

8. An automated method for preventing early termination of a laser-implemented surgical procedure on corneal tissue due to insufficient power, comprising:
determining a minimum amount of power required to perform a complete laser-implemented surgical procedure on a cornea;
storing the determination of the minimum amount of power required to perform the surgical procedure;
calculating an actual battery capacity based on the tested battery characteristic;
generating a control signal representing a comparison of the battery capacity to the stored determination of the amount of power required;
automatically preventing a refractive laser system from initiating the surgical procedure on the corneal tissue if the control signal falls below a minimum threshold value.

9. The method of claim 8, wherein the tested battery characteristic is a short-circuit current, and the short-circuit current is determined by short-circuiting the battery through a load.

10. The method of claim 8, wherein automatically preventing the refractive laser system from initiating the surgical procedure comprises interrupting power to the refractive laser system.

11. The method of claim 8, wherein the minimum amount of power required to perform the surgical procedure is the actual amount of power required to perform the surgical procedure.

12. The method of claim 8, wherein the minimum amount of power required to perform the surgical procedure is the actual amount of power required to perform the surgical procedure plus an additional predetermined reserve amount of power.

13. The method of claim 8, further comprising automatically alerting an operator that the battery capacity is less than the required minimum amount of power to perform the surgical procedure.

14. A power supply system for a refractive laser system, comprising:
a power supply module for supplying power to a refractive laser system under normal operating conditions;
an uninterruptible power supply in electrical communication with the power supply module and the refractive laser system, including:
a rechargeable battery for supplying power to the refractive laser system in the event that the power supply module fails,
a control module for determining an actual capacity of the rechargeable battery by short-circuiting the rechargeable battery through a test load and generating a control signal representing the actual battery capacity; and
wherein if the control signal is less than a minimum predetermined value, the uninterruptible power supply prevents the refractive laser system from operating.
